(19) United States
(12) Patent Application Publication
(10) Pub. No.: US 2006/0058599 A1
(43) Pub. Date: Mar. 16, 2006

(54) Assembly for storing and dispensing an electrode device

(75) Inventors: Edward A. Cummings, Armagh (GB); Bryan Overbury, Evesham (GB); Andrew Ledgeway, Fleet (GB); Alun Wilcox, Richmond (GB)

Correspondence Address: KLEIN, O'NEILL & SINGH, 2 PARK PLAZA, SUITE 510, IRVINE, CA, 92614, US

(73) Assignee: Unomedical Limited, Redditch (GB)

(21) Appl. No.: 11/228,001

(22) Filed: Sep. 15, 2005

Related U.S. Application Data
(60) Provisional application No. 60/610,855, filed on Sep. 17, 2004.

(30) Foreign Application Priority Data
Sep. 16, 2004 (EP) 04255614.2

(51) Int. Cl. A61B 5/04

(52) U.S. Cl. 600/391; 600/392

(57) ABSTRACT

An assembly for storing and dispensing an electrode device, of the kind that includes a sensor connected by a wire to a connector, includes a container for receiving the electrode device. The container defines a cavity having a dispensing aperture closed by a flexible cover. The cover is peelably attached to the container, and the sensor is releasably attached to an interior surface of the cover. The container advantageously includes a flange that extends outwardly from the cavity and that includes a groove that receives the wire. The cavity of the container advantageously contains a holder that receives the connector.

### CROSS-REFERENCE TO RELATED APPLICATION

This application claims the benefit, under 35 U.S.C. §119(e), of co-pending provisional application No. 60/610,855, filed Sep. 17, 2004, the disclosure of which is incorporated herein by reference.

### FEDERALLY SPONSORED RESEARCH OR DEVELOPMENT

Not Applicable

### BACKGROUND OF THE INVENTION

The present invention relates to a combination of a storage and dispensing assembly for storing and dispensing an electrode. Furthermore, the present invention relates to a set of storage and dispensing assemblies for storing and dispensing a set of electrodes. Still further, the present invention relates to a method for providing storage and dispensing of an electrode from a storage and dispensing assembly and a method for providing storage and dispensing of a set of electrodes from a set of storage and dispensing assemblies.

Storage and dispensing devices relating to the present invention are some times know as blister packs, and have been described in patent publications such as U.S. Pat. No. 5,390,792, U.S. 2003/0168370, U.S. 2003/0148110, U.S. Pat. No. 6,358,241, U.S. Pat. No. 6,183,467, U.S. Pat. No. 6,478,030, U.S. Pat. No. 5,814,043, U.S. 2002/0104770, U.S. 2002/082644, U.S. Pat. No. 5,462,457, U.S. Pat. No. 4,366,901, U.S. Pat. No. 5,050,732, EP 1 293 222 and U.S. 2003/0014040. Reference is made to all the above-mentioned US patent publications, all of which are hereby incorporated in the present specification by reference in their entirety for all purposes.

A conventional electrode device, such as a disposable electrode device for use in a monitoring system or measuring apparatus, e.g., an ECG monitoring system, comprises a sensor, an electrical wire and a connector for establishing electrically conductive connection to the monitoring or measuring apparatus. Generally, a problem exists in handling and properly storing the electrode devices and allowing an easy handling of the multiplicity of electrode devices without mixing the wires together and also keeping the wires or conductors of the individual electrode devices separate from one another without tying or mixing the conductors or wires together.

Conventionally, the electrode devices are stored in a state in which the sensor is fixated to a supporting foil, such as a plastic foil, and the wire or conductor of each of the electrode devices is wound up into a bundle. The handling of the individual electrode device in a monitoring or measuring situation may sometimes turn out to be inadequate and time consuming as the handling of the individual electrode device having the wire wound up in the bundles and the rewinding of the bundles before or after the electrode devices are applied in their intentional position on respective skin surface parts of a patient or person often tend to be tangled.

Therefore, there exists a need for providing a storage and dispensing assembly for storing and dispensing devices such as electrodes.

### SUMMARY OF THE INVENTION

According to a first aspect of the present invention, a combination of a storage and dispensing assembly for storing and dispensing an electrode is provided, the electrode being arranged in the assembly,
the electrode comprising an electrode head, a connecting means for connecting the sensor to a monitoring or measuring means, and a electrically conductive wire for connecting the electrode head to the connector,
the assembly comprising a container for receiving the electrode and having a dispensing aperture closed by a cover attached to the container by means of first releasable attachment means, the electrode head being releasably attached to a surface of the cover facing the interior of the container by means of second releasable attachment means.

The electrode may be placed in a receiver, e.g., a mechanical device for holding the electrode in a fixed position inside the container. In a preferred embodiment of the present invention, the second releasable attachment means comprises re-sealable adhesive suited for attachment of the electrode to human skin.

The adhesive first adheres the electrode to the covering means. When the assembly is opened, the person opening the assembly may remove the electrode head and apply the electrode to the intended surface. The adhesive may then ensure that the electrode is adhered to that intended surface. The intended surface may be the skin of a patient, such as an infant, a child, an adolescent, an adult or, alternatively an animal.

In one embodiment of the present invention, the electrically conductive wire may be a lead wire being transparent to radio waves.

A first feature of the present invention relates to the covering means comprising or being constituted by a flexible sheet and/or the first releasable attachment means being constituted by a peelable adhesive, such that the flexible sheet may be manually peeled away from the container. The flexible covering means may be a PE, PP, PVC foil, aluminum foil, a combination thereof, or a PE-AI-PE laminate or any other laminate. Alternatively, the first releasable attachment means may be constituted by heat welding. The container may define an extending flange or area to which the cover may be attached, by, e.g., heat welding or by means of an adhesive.

Alternatively, the covering means may comprise or be constituted by a rigid structure, comprising a lid, such as a plastic lid.

According to a second feature of the present invention, the container may be made from a plastic material, such as PE, PP or PVC, preferably being a transparent or translucent material, more preferably a material with a low moisture vapor transmission rate (MVTR). The transparent or translucent material allows a person to visually inspect the contents of the container. In the present context, the term “person” is to be construed as a generic term covering medical personnel, such as nurses and doctors, and any person that will apply an electrode stored in an assembly according to the present invention onto an intended surface.

A third feature of the present invention relates to the electrode head comprising a woven, breathable cloth, a medical grade polyethylene foam, and a silver, silver chloride, tin chloride or graphite sensing element.

According to a fourth feature of the present invention, the electrode head may define a gripping part. The gripping part may be a part of, e.g., the woven, breathable cloth or the polyethylene foam, or, alternatively, any other mechanically stable part of the electrode head.

The gripping part is contemplated to facilitate a person applying one or more of the electrode heads to an intended surface to separate the electrode head from the covering means. The gripping part may be formed as a handle, handgrip, projection or protruding part.

A fifth feature of the present invention relates to the container defining an open-faced cavity and a flange extending outward from the cavity, a circumferential groove formed in the flange, and the wire being received within the groove. The flange may extend from the container so that a plane is defined whereon the cover may be placed. The cover may be fastened to the flange or collar by means of adhesive, mechanical fastening, heat welding or the like, or alternatively any combinations thereof.

According to a sixth feature of the present invention, a holder may be formed in the open-faced cavity for receiving the connector. The holder is contemplated to ensure that the connector is not moved out of its position within the container, e.g. during storage and/or transport. Also, when a person removes the cover, the wire is also removed from the container and the holder is contemplated to keep the connector in place until the electrode head has been positioned correctly on the intended surface, provided that the person does not, intentionally or unintentionally, remove the connector from the container.

A seventh feature relates to the container defining a cavity, the wire being wound in a loop, the loop being received in the cavity. The wire may be coiled or wound into a loop, which may then be placed into the cavity. This enables a machine or person to wind, spin, coil or loop the wire so that the wire may be received within the cavity. When the wire loop is then released inside the cavity, the wire is contemplated to unwind and press against the sidewalls of the cavity, thereby holding itself in place. Alternatively, the loop may be supported by a body, the body and the loop then being received in the cavity. Supporting the wire loop with a body enables the wire to be tightly wound around the body, and possibly fixing the wire to the body so that the wire will not unwind after placing the body and wire in the cavity.

Advantageously, the body may define a circumferential groove wherein the wire is received. The risk of the wire then unwinding from the body during storage, transport or dispensing is contemplated to be reduced.

According to the teachings of the present invention, the container may define an open-faced cavity, and a holder may be formed in the open-faced cavity for receiving the connector. The holder is contemplated to ensure that the connector does not exit the cavity during the application or dispensing of the electrode device.

According to a second aspect of the present invention, a set of storage and dispensing assemblies for storing and dispensing a set of electrodes is provided, such an electrode being arranged in each of the assemblies,
each of the electrodes comprising a sensor, a connecting means for connecting the sensor to a monitoring or measuring means, and a electrically conductive wire for connecting the sensor to the connector,
each of the assemblies comprising a container for receiving a respective electrode and having a dispensing aperture closed by a cover attached to the container by means of first releasable attachment means, the sensor being releasably attached to a surface of the cover facing the interior of the container by means of second releasable attachment means.

Persons using the storage and dispensing devices according to the present invention may require placing a number of electrodes on the intended surface. The number of electrodes may be 2, 3, 4, 5 or even more. Therefor, the present invention provides the set of storage and dispensing devices according to the second aspect of the present invention. Each of the assemblies of the set of storage and dispensing assemblies may further comprises any of the features of the storage and dispensing device according to the first aspect of the present invention.

According to a third aspect of the present invention, a method for providing storage and dispensing of an electrode from a storage and dispensing assembly is provided. The method according to the third aspect may comprise the steps of:
providing said electrode,
said electrode device comprising a sensor, a connector for connecting said sensor to a monitoring or measuring means, and a electrically conductive wire for connecting said sensor to said connector,
providing an assembly, said assembly comprising a container for receiving said electrode device and having a dispensing aperture closed by a cover attached to said container by means of first releasable attachment means,
arranging said electrode device in said assembly, and
releasably attaching said sensor to a surface of said cover facing the interior of said container by means of second releasable attachment means.

It is a first object of the present invention that the method according to the third aspect, wherein the container defines an open faced cavity and a flange extending outward from the cavity, may further comprise the steps of:
providing a circumferential groove formed in the flange, and
positioning the wire within the groove.

Providing a groove is contemplated to provide an orderly storage of the wire within the container. Also, it is contemplated that when applying the electrode to the intended surface, the wire will unravel in a orderly fashion, so that the wire does not get tangled or otherwise hindered.

A second object of the present invention relates to
providing a holder in the container, and
positioning the connector in the holder.

A holder is contemplated to provide an improved storage of the electrode connector during transport and/or storage. Also, during the application of the electrode to an intended surface, the connector is held within the container, and it is contemplated that this will reduce the risk of damaging the connector unintentionally.

A third object of the present invention, relates to the method according to the third aspect, further comprising:
providing a gripping part in the cover for allowing a person to at least partly separate the cover from the container.

A gripping part or handle, is contemplated to provide an improved grip for a person removing the cover of the assembly.

A fourth object of the present invention relates to providing a gripping part in the sensor so as to allow a person to detach the sensor from the covering means.

A gripping part or handle is contemplated to provide an improved grip for a person removing the sensor from the covering means of the assembly, while the part of the sensor intended for being in facial contact with an intended surface is not touched and thereof the risk of possible contamination is contemplated to be reduced.

The devices used in connection with the method according to the third aspect of the present invention may incorporate any of the features in connection with the first aspect relating to a storage and dispensing device.

A fourth aspect of the present invention provides a method for providing storage and dispensing of a set of electrodes from a set of storage and dispensing assemblies, that may comprise the steps of:
arranging each of the electrode devices in a specific assembly,
each of the electrode devices comprising a sensor, a connector for connecting the sensor to a monitoring or measuring means, and an electrically conductive wire for connecting the sensor to the connector,
each of the assemblies comprising a container for receiving the electrode device and having a dispensing aperture closed by a covering means attached to the container by means of first releasable attachment means,
releasably attaching each of the sensors to a surface of a specific cover facing the interior of a specific container by means of second releasable attachment means.

After providing a plurality of storage and dispensing devices, sensors including an electrode head, a wire, and a connector may be arranged in the assembly.

A seventh feature of the present invention relates to the covering means being constituted by a set of covers, where each cover seals or closes a specific container. The method may then further comprise removing a specific cover of the set for exposing a specific sensor and/or container. The cover may be removed from a particular container for extracting or removing a specific sensor stored in that particular container.

An eight feature of the method according to the present invention relates to the covering means being constituted by a unitary covering means. The cover may be constituted by a single piece of material, such as a foil or laminate, covering the entire set of assemblies. The containers may as well be connected so that a person applying electrodes to intended surfaces may hold one package comprising the desired number of electrodes at once. The set of assemblies may alternatively be separated by a health care person into individual assemblies.

According to a ninth feature of the present invention, the method may comprise the additional step of removing a specific cover for exposing a single specific electrode or removing the cover for exposing the set of electrodes.

The cover may be crafted so that it is possible for a person to remove the cover from a specific container without exposing the remaining containers. This may be achieved by, e.g. peeling the cover in a first direction. A person may alternatively remove the entire cover in one operation, e.g. by peeling the covering means in a second direction, different from the first direction, so that all of the containers are exposed, substantially at once.

The method according to the fourth aspect of the present invention may incorporate any of the features relating to the first, second or third aspect.

A fifth aspect of the present invention relates to a set of storage and dispensing assemblies, each assembly comprising a container, wherein at least one device is arranged, each of the containers having a dispensing aperture closed by a flexible cover attached to the container by means of a releasable attachment means, at least one thinned area defined in the flexible cover, the flexible cover being peelably detachable from the assembly in at least a first and a second direction so that when the flexible cover is peeled from a first direction, the flexible cover is removed from one specific container, and when the flexible cover is peeled from a second direction, the flexible cover is removed from all of the containers.

In the set of storage and dispensing assemblies covered by the flexible cover, a person opening the assembly may need to open only one of the assemblies in an operation, e.g. for attaching an electrode to a skin part of a patient, and repeating this operation for each of the electrodes stored in the assembly. Alternatively, the person may need to remove the cover from all of the containers in one single operation while still having the electrode devices attached to the cover for, e.g., application to the skin surface part of the patient.

As discussed above, the electrode device includes a wire and a connector for connecting to an apparatus for performing monitoring and measuring operations, and the wire and/or connector may be rolled up on a supporting body or in a coil positioned in a groove within the container.

The thinned area may be constituted by an area or line where the sheet constituting the flexible cover includes perforations or a lesser amount of material compared to the surrounding areas.

The first and the second direction may, e.g., be from two corners of a package or assembly. If the assembly or package is rectangular or square, the first direction and the second direction may be from corners on the same side, e.g. the short side of the rectangle or from two corners in place diagonally from each other.

### BRIEF DESCRIPTION OF THE DRAWINGS

The present invention is now to be described in more detail with reference to the drawings, in which:

FIG. 1 is a perspective view of a storage and dispensing assembly, in accordance with the present invention;

FIG. 2 is an idealized view of an electrode device separated from an assembly of the type shown in FIG. 1;

FIG. 3 is a view of an assembly line for assembling storage and dispensing assemblies in accordance with the present invention;

FIG. 4 is a top plan view of a pack comprising three storage and dispensing assemblies in accordance with the present invention;

FIG. 5 is a perspective view of a body supporting a wire loop for use in the present invention; and

FIGS. 6a through 6e are schematic illustrations of a process of winding a wire into a loop.

### DETAILED DESCRIPTION OF THE INVENTION

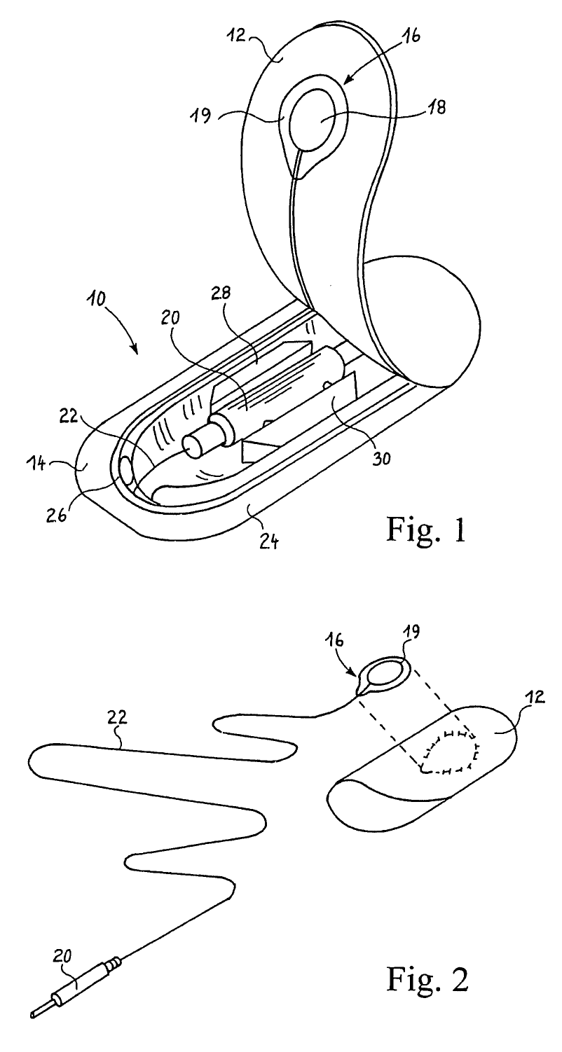

FIG. 1 is a view of a combination of a storage and dispensing assembly for storing and dispensing an electrode device. The storage and dispensing assembly is in its entirety designated the reference numeral 10. The assembly 10 comprises a cover 12, preferably being a flexible plastic material foil or a flexible aluminum foil. The cover 12 is attached to a container 14. The cover is preferably heat sealed to the container 14 and may be peeled from the container 14.

An electrode device 16 is arranged inside the assembly 10, preferably fixed using a releasable adhesive, so that a person, such as a nurse, doctor, or other health care personnel, may remove the electrode device 16 from the cover 12 and position the electrode device 16 on an intended surface, such as a skin part of a patient. The electrode device 16 comprises a sensor 18, positioned in a sensor head 19. The sensor 18 preferably is an electrically conductive element, such as a silver, silver chloride, tin chloride or graphite sensing element. The sensor 16 further comprises a connector 20 for establishing a connection to a monitoring or measuring apparatus or system (not shown), and an electrically conductive wire 22 for establishing an electrical connection between the sensor 18 and the connector 20.

The container 14 defines a cavity wherein the connector 20, the wire 22 and the sensor head 19 is received. The container 14 further defines a flange 24 extending outwardly from the container 14. In the flange 24, a circumferential groove 26 is defined. The wire 22, or a part thereof, is preferably arranged within the groove 26, so that when a health care person applies the electrode device 16 to a patient, or a skin part of a patient, the wire 22 does not get tangled.

The connector 20 may be received within a holder, which, in the presently preferred embodiment of the present invention, comprises two protruding parts 28 and 30 for fixing the connector 20 during storage and/or transport, and also during application of the electrode device 16 to the intended surface.

FIG. 2 is a view of the electrode device 16 separated from the assembly 10 and the sensor head 19 detached from the cover 12.

In the presently preferred embodiment of the present invention, the sensor head 19 comprises two central fabric bodies, whereon a sensor electrode is mounted. The sensor 16 is applied with an adhesive, preferably an electrically conductive hydrogel or other skin-friendly adhesive. A lead wire assembly establishes the electrical connection from the sensor to the monitoring or measuring or apparatus. A foam cover may be applied on top of the lead wire and an upper fabric body.

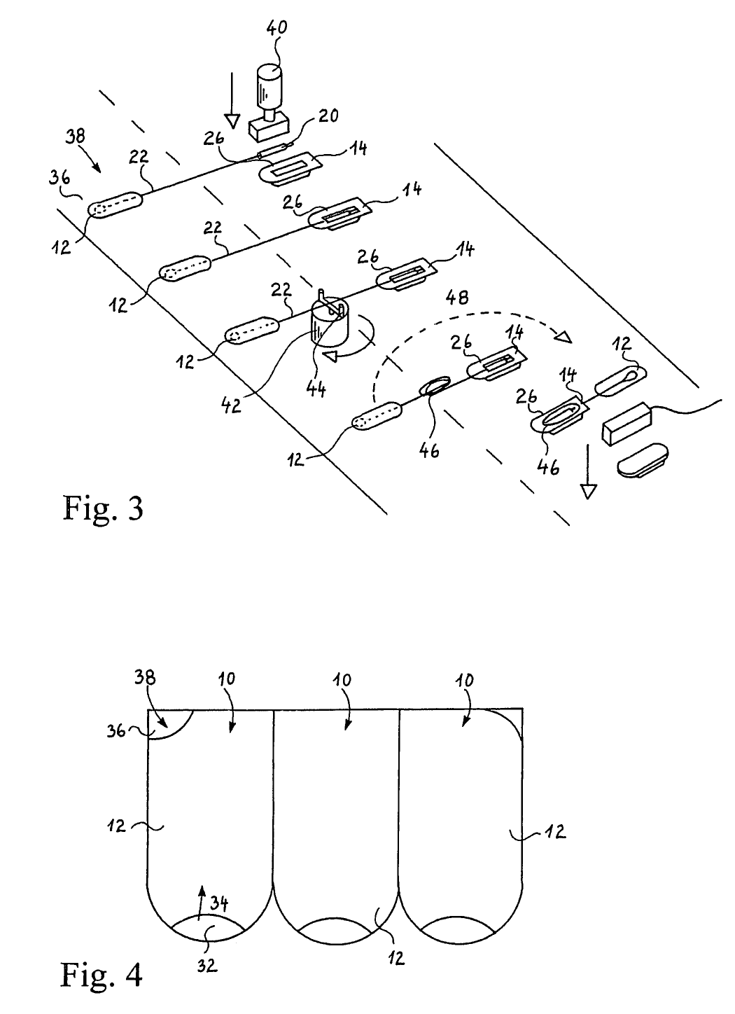

FIG. 3 is a schematic illustration of an assembly line for assembling covers 12 and containers 14. The covers travel on a conveyor 36 in the direction of the arrow 38. The conveyor may include a laminate whereon the individual covers are formed, e.g. by cutting or stamping a laminate. Also, an adhesive for adhering the covers 12 to the containers 14 may be applied to the covers 12.

The releasable and skin friendly adhesive is applied to either the electrode device 16 or to the cover 12 for fixing the sensor head 19 to the inside of the cover 12. A machine 40 places the connector 20 in the container 14, specifically between the protruding parts 28 and 30 in the cavity of a restrictive container. Prior to this, the container 14 may be stamped from a substrate by a stamping machine, thereby forming the individual parts of the container 14, such as the cavity, the flange 24, the groove 26 and the protruding parts 28 and 30.

A tool 42 for winding the wire 22 into a loop is also placed on or near the conveyor 36. The tool 42 preferably includes a rotational part 44 that grips the wire 22 and winds the wire 22 into a loop 46. Afterwards, the looped wire 46 is positioned in the cavity of the container, specifically and preferably into the groove 26 of the container 14. This may be achieved by moving the cover 12 in the direction of the arrow 48 so as to place or position the loop 46 substantially above or in the groove 26. Finally, the cover 12 may be positioned on the flange 24 and fixed to the flange, e.g., by heat welding or by an adhesive.

The order of steps performed on the assembly line is not to be considered limited to the order described in the present description. It might be contemplated to be advantageous to perform certain steps before or after other steps; for instance, the winding of the wire 22 may be performed during the positioning of the wire 22 in the container 14 or the groove 26, and the positioning of the connector 20 in the holder in the container 14 may be performed after the winding of the wire 22.

The covers 12 may be cut or stamped from a substrate or laminate at any point, before, after, or during positioning of the electrode device 16.

FIG. 4 is a top view of three assemblies supplied as one package. A health care person is thereby able to apply three electrodes to an intended surface without having to leave the patient for acquiring more electrodes. Even though FIG. 4 illustrates a package with three assemblies, two, four or even more assemblies may be assembled into a single package.

When the health care person is to apply the individual electrode devices 16, the health care person may desire to expose only one electrode device at one time or all of the electrode devices in the package. The health care person may choose to remove a single cover 12 by pulling the cover at the area designated 32 in the direction of the arrow 34. Alternatively, the health care person may pull the cover in the area 36 in the general direction of the arrow 38, thereby exposing multiple electrode devices at one time.

The cover 12 may include one or more thinned, attenuated or perforated areas, constituting one or more break lines, for enabling a health care person to expose only one electrode device one time. The health care person then pulls the cover 12 in a first direction for exposing one electrode. Alternatively, the health care person pulls the cover 12 in a second direction for exposing multiple, or all, of the electrode devices at one time. The break lines may be formed by stamping, cutting or punctuating the cover 12. Alternatively, the break lines may be formed in one or more layers of a laminate constituting the cover 12.

FIG. 5 is a schematic view of a body 54 supporting a wire loop 56. A wire is wound into a loop 56 for facilitating the positioning of the wire in the cavity of a dispensing assembly. In the embodiment illustrated in FIG. 5, the wire loop 56 is received in a groove 58 defined in the body 54. The body 54 illustrated in FIG. 5 is large enough to receive a connector 60 in a holder 62 defined in the body 54. However, embodiments where a connector 60 is not received in a holder in the body are also possible. Furthermore, the connector may be received in an aperture defined in the body.

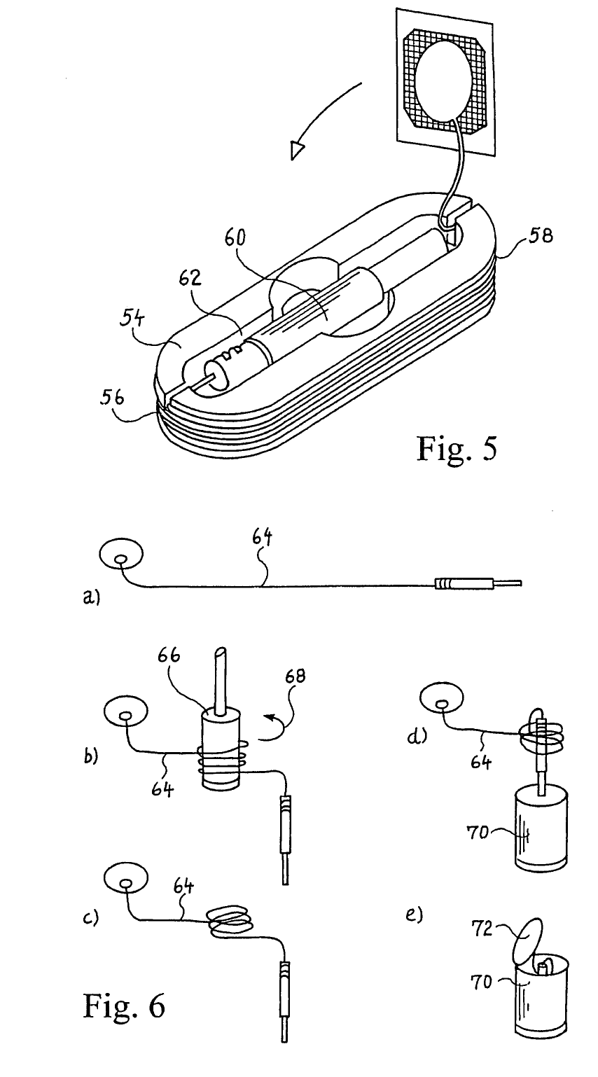

FIGS. 6a through 6e are schematic illustrations of a process of winding a wire 64 into a loop.

In FIG. 6a, the unwound wire 64 is illustrated. In FIG. 6b, a tool 66 grabs the wire 64 at a point, preferably near or at the center or middle of the wire 64. The tool 66 may then roll or wind the wire 66 into a loop, e.g. by rotating in the direction of the arrow 68.

The tool 66 may be removed, provided that the loop is maintained despite the lack of support, as shown in FIG. 6c. If it is not possible to remove the tool 66 without causing the loop to unwind, the tool 66 is not removed, and the step shown in FIG. 6c is not performed. The wound wire 64 may then be placed or received in an appropriate container 70, as illustrated in FIG. 6d. The wire loop may be supported by a body, such as that illustrated in FIG. 5, or the wire loop may be unsupported. Finally, as shown in FIG. 6e, the container 70 may be sealed or closed by, e.g. a lid 72, possibly comprising a flexible cover, as discussed above in relation to FIGS. 1-4.

What is claimed is:

1. A combination of an electrode device and a storage and dispensing assembly for storing and dispensing the electrode device, the combination comprising:
said electrode device comprising a sensor, a connector for connecting said sensor to a monitoring or measuring means, and an electrically conductive wire for connecting said sensor to said connector; and
said assembly comprising a container for receiving said electrode device and having a dispensing aperture closed by a cover that is releasably attached to said container;
wherein said sensor is releasably attached to a surface of said cover facing the interior of said container.

2. The combination according to claim 1, wherein said container defines a cavity and a flange extending outward from said cavity, said flange including a circumferential groove, in which said wire is received.

3. The combination according to claim 2, wherein a holder is formed within said cavity of said container, in which holder said connector is received.

4. The combination according to claim 1, wherein said container defines a cavity, said wire being wound in a loop, said loop being received in said cavity.

5. The combination according to claim 4, wherein said loop is supported by a body, and wherein said body and said loop are received in said cavity.

6. The combination according to claim 5, wherein said body defines a circumferential groove wherein said wire is received.

7. The combination according to claim 4, wherein a holder is formed within said cavity of said container, in which holder said connector is received.

8. The combination according to claim 1, wherein said sensor is attached to said surface of said cover by a re-sealable adhesive.

9. The combination according to claim 8, wherein said resealable adhesive comprises an electrically conductive hydrogel.

10. The combination according to claim 1, wherein said cover comprises a flexible sheet, and wherein said cover is releasably attached to said container by a peelable adhesive, such that said flexible sheet may be manually peeled away from said container.

11. The combination according to claim 1, wherein said cover comprises a foil made from a material selected from the group consisting of one or more of PE, PP, PVC, aluminum, and a combination thereof.

12. The combination according to claim 1, wherein said cover is attached to said container by heat welding.

13. The combination according to claim 1, wherein said container is made from a plastic material selected from the group consisting of at least one of PE, PP and PVC.

14. The combination according to claim 1, wherein said sensor defines a gripping part.

15. The combination according to claim 1, wherein said sensor comprises a woven breathable cloth, a medical grade polyethylene foam, and a sensing element that includes a material selected from the group consisting of at least one of silver, silver chloride, tin chloride and graphite.

16. A set of storage and dispensing assemblies for individually storing and dispensing electrodes, characterized in that:
each of said electrodes devices comprises a sensor, a connector for connecting said sensor to a monitoring or measuring means, and an electrically conductive wire for connecting said sensor to said connector; and
each of said assemblies comprises a container for receiving a respective electrode device and having a dispensing aperture closed by a cover that is releasably attached to said container, and wherein said sensor is releasably attached to a surface of said cover facing the interior of said container.

17. A method for providing storage and dispensing of an electrode device from a storage and dispensing assembly, comprising the steps of:
providing said electrode device comprising a sensor, a connector for connecting said sensor to a monitoring or measuring means, and an electrically conductive wire for connecting said sensor to said connector;
providing an assembly comprising a container for receiving said electrode device and having a dispensing aperture closed by a cover that is releasably attached to said container;
arranging said electrode device in said assembly; and
releasably attaching said sensor to a surface of said cover facing the interior of said container.

18. The method according to claim 17, wherein said container defines a cavity and a flange extending outward from said cavity, the method further comprising:
providing a circumferential groove formed in said flange; and
positioning said wire within said groove.

19. The method according to claim 18, further comprising:
providing a holder within said cavity; and
positioning said connector in said holder.

20. The method according to claim 17, further comprising:
providing a gripping part in said cover for allowing a person to at least partly separate said cover from said container.

21. The method according to claim 17, further comprising:
providing a gripping part at said sensor so as to allow a person to detach said sensor from said cover.

22. A method for providing storage and dispensing of a set of electrode devices from a set of storage and dispensing assemblies, comprising:
arranging each of said electrode devices in a specific assembly, each of said electrode devices comprising a sensor, a connector for connecting said sensor to a monitoring or measuring means, and an electrically conductive wire for connecting said sensor to said connector, each of said assemblies comprising a container for receiving said electrode device and having a dispensing aperture closed by a cover that is releasably attached to said container; and
releasably attaching each of said sensors to a surface of a specific cover facing the interior of a specific container.

23. The method according to claim 22, wherein said cover comprises a set of covers, the method further comprising:
removing a specific cover of said set for exposing a specific electrode.

24. The method according to claim 22, wherein said cover comprises a unitary cover, the method further comprising at least one of:
removing a specific cover for exposing a single specific electrode device; and
removing said cover for exposing said set of electrode devices.

25. A set of storage and dispensing assemblies, each assembly comprising a container, wherein at least one device is arranged in the container, each of said containers having a dispensing aperture closed by a flexible cover attached to said container by releasable attachment means, at least one thinned area being defined in said flexible cover, said flexible cover being peelably detachable from said assembly in at least a first and a second direction, so that when said flexible cover is peeled in said first direction, said flexible cover is removed from one specific container, and when said flexible cover is peeled in said second direction, said flexible cover is removed from all of said containers.

26. The set of storage and dispensing assemblies according to claim 25, wherein said flexible cover is made of a foil material selected from the group consisting of at least one of PE, PP, PVC, aluminum, and a combination thereof.
